(19) United States
(12) Patent Application Publication
(10) Pub. No.: US 2023/0348819 A1
(43) Pub. Date: Nov. 2, 2023

(54) USE OF ESSENTIAL OILS FOR REDUCING OR PREVENTING MALODOR IN FABRICS, TEXTILES OR CLOTHING

(71) Applicant: Vistobio-Comercio de Perfumes Ltda., São Paulo (BR)

(72) Inventors: Renan Nacco Galindo SERRANO, São Bernardo (BR); Chris CALLEWAERT, Gent (BE)

(73) Assignee: Vistobio-Comercio de Perfumes Ltda., São Paulo (BR)

(21) Appl. No.: 17/918,631

(22) PCT Filed: Apr. 13, 2020
(86) PCT No.: PCT/BR2020/050125

§ 371 Date: Oct. 13, 2022

(51) Int. Cl. C11D 3/00; C11D 3/50; D06M 13/00; D06M 23/12; C11D 3/382; A61L 2/18; A61L 2/232; D06M 16/00

(52) U.S. Cl. CPC C11D 3/0068; A61L 2/18; A61L 2/232; C11D 3/382; C11D 3/505; D06M 13/005; D06M 16/00; D06M 23/12; A61L 2202/26

(57) ABSTRACT

The present invention relates to the field of reducing clothing malodor which is due to bacterial conversion of molecules which are present in sweat. The present invention discloses micro-encapsulated essential oils that reduces malodor on clothes in a durable way. The compounds of the present invention can thus be used in clothing sprays, clothing finishing agents, deodorants, washing powders, or in any method to reduce laundry malodor.

### FIELD OF THE INVENTION

The present invention relates to the field of reducing laundry malodor which is due to bacterial conversion of molecules which are present in sweat. The present invention discloses micro-encapsulated essential oils that reduces malodor on clothes in a durable way. The compounds of the present invention can thus be used in clothing sprays, clothing finishing agents, deodorants, washing powders, or in any method to reduce malodor.

### BACKGROUND OF THE INVENTION

Clothing textiles protect the human body against external factors, such as UV, chemicals, cold and heat. The textiles are in close contact with the skin, and as such, the microorganisms of the skin can easily be transferred to the textiles. The textiles are not sterile and can contain high bacterial amounts, particularly on warm and moist areas of the body (e.g. underarm, groin, and elsewhere). In some cases, the textiles can create a warm and often moist environment on the skin, which leads to an excess of bacteria. The bacteria, coming from the skin and present on fresh clothing, are able to grow on the sweat secretions. The moisture, lipids, amino acids, breakdown products from skin keratinocytes and others help in growing a bacterial biomass. In some cases, the growth of the bacteria on the textiles leads to unpleasant odors, staining, fabric deterioration and even physical irritation (Szostak-Kotowa 2004). The most known problem is the case of textile malodor, particularly in the underarm region (Chris Callewaert, De Maeseneire, et al. 2014). Axillary malodor does not only come from the skin but also from the textiles near the axillary region (Dravniek et al. 1968; Shelley, Hurley, and Nicholas 1953). Primary underarm odor is described as the odor emitted directly from the axillary skin, while secondary underarm odor is described as odor coming from clothing in contact with the axilla (Dravniek et al. 1968). The odor can significantly differ between skin and textile (McQueen et al. 2007). Research showed that polyester and synthetic clothes smelled significantly less pleasant as compared to cotton clothes (Chris Callewaert, De Maeseneire, et al. 2014).

Most of the microbiota living on the skin and textiles are, however, harmless. A handful of bacteria are known to be linked with production of malodors. Many other bacteria are not associated with malodors or are even associated with good odors (Myriam Troccaz et al. 2015). In the case of textile malodor, the textile microbiome is known to play an important role, and is considered, at least partially, responsible for the malodor formation (Chris Callewaert, De Maeseneire, et al. 2014). Treatment is usually done with deodorant, which are aimed to be used on skin. To date however, there is no long-lasting solution focusing on malodor generation on clothes. Deodorants contain perfumes, to mask the malodor production. They also contain antimicrobial compounds to limit the bacterial growth. Compounds with an antimicrobial and/or antifungal function that are commonly used are triclosan, triclocarban, quaternary ammonium compounds, metal salts, aliphatic alcohols, glycols and other fragrances (Makin and Lowry 1999) (Boonme and Songkro 2010). These compounds are from chemical and synthetic origin and can have a vast, persistent and detrimental effect on the skin microbiome and metabolome (Bouslimani et al. 2019). Many of these synthetic and cosmetic ingredients lead to a microbial shock on skin and clothes and lead to an increase in microbial diversity (Chris Callewaert, Hutapea, et al. 2014). This is unwanted as a higher microbial diversity is associated with a stronger underarm malodor. The abundance of corynebacteria may increase, which can lead to more malodor formation (Chris Callewaert, Hutapea, et al. 2014).

Synthetic antimicrobials and antibiotics have long and widely been used in medicine and for topical and cosmetic purposes. A big drawback is the growing antimicrobial resistance which makes the treatments less effective (Schelz, Molnar, and Hohmann 2006). The synthetic and active ingredients are pure and are an easy target for antibacterial resistance genes with microorganisms, on or near mobile genetic elements.

For these reasons, alternative, natural and mixed active ingredients have gained much attention for use as cosmetic application. Plants and other natural sources have been shown to provide a great range of complex and structurally diverse compounds. Plant extracts and essential oils were shown to possess antifungal, antibacterial, and antiviral properties and have been screened on a global scale as potential sources of novel antimicrobial compounds, agents promoting food preservation, and alternatives to treat infectious diseases (Safaei-Ghomi, J., & Ahd, A. A. 2010).

Essential oils have also been reported to possess significant antiseptic, antibacterial, antiviral, antioxidant, anti-parasitic, antifungal, and insecticidal activities. They have gained interest, especially when the actives were obtained from plants (O′Bryan et al. 2015; Prabuseenivasan, Jayakumar, and Ignacimuthu 2006). Various essential oils of different plants such as thyme, oregano, mint, cinnamon, cumin, salvia, clove, and eucalyptus have been observed to possess strong antimicrobial properties (Sienkiewicz et al. 2014). Additionally, these essential oils have a natural fragrance, containing a wide mixture of natural scents. In addition, a wide range of essential oils possess antioxidant properties. For this purpose, we proposed the use of natural essential oils that have the capability to release a good fragrance, as well as have a bacteriostatic effect on the microbiome.

Essential oils such as Tea Tree oil (Melaleuca alternifolia) (Carson, Hammer, and Riley 2006), Clove oil (Eugenia caryophyllus) (Rajkumar and Berwal 2003), Thyme oil (Thymus vulgaris) (Rota et al. 2008), Lemongrass oil (Cymbopogon flexuosus) (Adukwu et al. 2016), Cinnamon oil (Cinnamomum zeylanicum) (Unlu et al. 2010), Lavender oil (Lavandula officinalis) (Shafaghat, Salimi, and Amani-Hooshyar 2012), and Lavandin oil (Lavandin abrialis) (N′dédianhoua et al. 2014) are known to exhibit strong antimicrobial effect, both against bacteria, fungi and yeasts. Also, Sandalwood oil (Jirovetz et al. 2006) and Patchouli oil (Yang et al. 2013) are known to possess antimicrobial properties. Some of these essential oils have been used in traditional Chinese medicine (Yang et al. 2013). Many tests have shown the potency of essential oils to inhibit the growth of pathogenic bacteria (Escherichia coli, Pseudomonas aeruginosa, Bacillus proteus, Shigella dysenteriae, Typhoid bacillus, Staphylococcus aureus), as well as pathogenic fungi (Penicillium, Aspergillus, Candida).

Essential oils are a complex natural mixture and may consist of up to 60 components at different concentrations. Essential oils usually consist of two or three major components being present at high concentrations (20-70%) and several other components that may be present in trace amounts. The amount of the different components of essential oils varies amongst different plant parts and different plant species as they are chemically derived from terpenes and their oxygenated derivatives i.e., terpenoids that are aromatic and aliphatic acid esters and phenolic compounds. An important characteristic of essential oils and its components is their hydrophobicity, which enables them to partition with the lipids present in the cell membrane of bacteria and mitochondria, rendering them more permeable by disturbing the cell structures (Devi, K.P. et al. 2010).

Several different plant-derived essential oils have been studied for their antibacterial and antifungal characteristics.

Tea tree oil, the volatile essential oil derived mainly from the Australian native plant Melaleuca alternifolia. It is employed largely for its antimicrobial properties, tea tree oil is incorporated as the active ingredient in many topical formulations used to treat cutaneous infections (Carson, Hammer, and Riley 2006).

The essential oil from leaves and flowers of lavender (Lavandula officinalis) also showed important antibacterial activity. It has been shown that different concentrations of Lavandula officinalis essential oil have significant effect in the inhibition of at least three bacteria strains (Staphylococcus aureus, Klebsiella pneumonia and Pseudomonas aeruginosa) using disk diffusion method (Gavanji, S. et al 2014).

Essential oil of cinnamon (Cinnamomum zeylanicum bark oil) has also been shown to have potential anti-microbial action against a wide variety of bacteria (Acinetobacter baumannii, Acinetobacter lwoffii, Bacillus cereus, Bacillus coagulans, Bacillus subtilis, Brucella melitensis, Clostridium difficile, Clostridium perfringens, Enterobacter aerogenes, Enterobacter cloacae, Enterococcus faecalis, Enterococcus faecium, Escherichia coli, Haemophilus influenzae, Helicobacter pylori, Klebsiella pneumonia, Listeria ivanovii, Listeria monocytogenes, Mycobacterium smegmatis, Mycobacterium tuberculosis, Proteus mirabilis, Pseudomonas aeruginosa, Saccharomyces cerevisiae, Salmonella typhi, Salmonella typhimurium, Staphylococcus albus, Staphylococcus aureus, Streptococcus agalactiae, Streptococcus pneumoniae, Streptococcus pyogenes and Yersinia enterocolitica). In addition there also seems to be activity against numerous fungi (Aspergillus flavus, Aspergillus fumigatus, Aspergillus nididans, Aspergillus niger, Aspergillus ochraceus, Aspergillus parasiticus, Aspergillus terreus, Candida albicans, Candida glabrata, Candida krusei, Candida parapsilosis, Candida tropicalis, Cryptococcus neoformans, Epidermophyton floccosum, Histoplasma capsulatum, Malassezia furfur, Microsporum audouini, Microsporum canis Microsporum gypseum, Trichophyton mentagrophytes, Trichophyton rubrum and Trichophyton tonsurans) (Ranasinghe, P. et al. 2013).

Other essential oils from different less investigated plants, such as those from the Eugenia genus was determined against six common nosocomial pathogens, namely: Gram-positive bacteria (Bacillus cereus, Enterococcus faecalis, Staphylococcus aureus), Gram-negative bacteria (Escherichia coli, Pseudomonas aeruginosa, Salmonella Typhimurium) (Famuyide, I. M. et al., 2019). Plants from the Juniperus genus have also shown antibacterial activity alone and in combination with other essential oils (Rapper, S. et al., 2013),

Oil from Thymus vulgaris has also been shown to have strong inhibitory activity on some pathogens, including S. pyogenes, S. mutans, C. albicans, A. actinomycetemcomitans, and P. gingivalis as measured by agar disk diffusion and broth microdilution methods (Fani, M. and Kohanteb, J., 2017). Potent antifungal activity of the essential oils of Thymus on Candida spp. was also shown (Pina-Vaz C. et al., 2004). Upon today, there is a vast need for natural and alternative products and methods to reduce textile malodor.

Thus, it is an object of the present invention to provide a method of inhibiting or reducing textile malodor, with excellent efficacy, excellent cosmetic properties and aesthetics, reduced or no skin irritation and no fabric damage.

### BRIEF DESCRIPTION OF THE DRAWINGS

FIG. 1. Morphology and size of the nanoparticles using Scanning Electron Microscope (SEM), operated at 5 kV. (a) Display of about 50 µm of nanoparticles in aqueous solution. (b) Display of about 25 µm of nanoparticles in aqueous solution.

FIG. 2. Interaction of the nanoparticles on clothing textiles using Scanning Electron Microscope (SEM), operated at 1.5 kV. (a) Display of 144 µm of nanoparticles on cotton textile fibers. (b) Display of 144 µm of nanoparticles on cotton textile fibers at a different position.

FIG. 3. Bacterial MIC test results. Die-off of bacteria was noticed from a concentration of 10 µg/mL onwards.

FIG. 4. Odor panel results after 24 h of wearing of treated versus untreated textile samples. The treatments show a significant improvement in hedonic value of treated textiles versus untreated textiles.

### BRIEF DESCRIPTION OF THE INVENTION

In one aspect, the invention provides the use of at least one essential oil to reduce or to prevent malodor in fabrics, textiles or clothing.

In one embodiment, at least one essential oil is in the form of microparticles.

In a further embodiment, at least one essential oil is in the form of nanoparticles.

In another embodiment, at least one essential oil is contained in nano capsules.

In a further embodiment, the essential oil is selected from sesame oil, pyrethrum, glycerol-derived lipids or glycerol fatty acid derivatives, cinnamon oil, cedar oil, clove oil, geranium oil, lemongrass oil, angelica oil, mint oil, turmeric oil, wintergreen oil, rosemary oil, anise oil, cardamom oil, caraway oil, chamomile oil, coriander oil, guaiac wood oil, cumin oil, dill oil, mint oil, parsley oil, basil oil, camphor oil, cananga oil, citronella oil, eucalyptus oil, fennel oil, ginger oil, copaiba balsam oil, perilla oil, cedarwood oil, jasmine oil, palmarosa sofia oil, western mint oil, star anise oil, tuberose oil, neroli oil, tolu balsam oil, patchouli oil, palmarosa oil, Chamaecyparis obtusa oil, Hiba oil, sandalwood oil, petitgrain oil, bay oil, vetiver oil, bergamot oil, Peru balsam oil, bois de rose oil, grapefruit oil, lemon oil, mandarin oil, orange oil, oregano oil, lavender oil, Lindera oil, pine needle oil, pepper oil, rose oil, iris oil, sweet orange oil, tangerine oil, tea tree oil, tea seed oil, thyme oil, thymol oil, garlic oil, peppermint oil, onion oil, linaloe oil, Japanese mint oil, spearmint oil, linalool, limonene, geraniol, citral, eugenol, or combinations thereof.

In a preferred embodiment, the essential oil is a combination of two or more essential oils, selected from Melaleuca alternifolia oil, Lavandula officinalis oil, Cinnamomum zeylanicum oil, Eugenia caryophyllus oil, Cymbopogon flexuosus oil, Juniperus virginiana oil, Thymus vulgaris oil, or combinations thereof.

In another aspect, the invention provides a formulation comprising at least one essential oil and a vehicle for use in reducing and/or preventing malodor in fabrics, textiles or clothing.

In one embodiment, at least one essential oil is in the form of microparticles.

In a further embodiment, at least one essential oil is in the form of nanoparticles.

In another embodiment, at least one essential oil is contained in nano capsules.

In one embodiment, the essential oil is selected from sesame oil, pyrethrum, glycerol-derived lipids or glycerol fatty acid derivatives, cinnamon oil, cedar oil, clove oil, geranium oil, lemongrass oil, angelica oil, mint oil, turmeric oil, wintergreen oil, rosemary oil, anise oil, cardamom oil, caraway oil, chamomile oil, coriander oil, guaiac wood oil, cumin oil, dill oil, mint oil, parsley oil, basil oil, camphor oil, cananga oil, citronella oil, eucalyptus oil, fennel oil, ginger oil, copaiba balsam oil, perilla oil, cedarwood oil, jasmine oil, palmarosa sofia oil, western mint oil, star anise oil, tuberose oil, neroli oil, tolu balsam oil, patchouli oil, palmarosa oil, Chamaecyparis obtusa oil, Hiba oil, sandalwood oil, petitgrain oil, bay oil, vetiver oil, bergamot oil, Peru balsam oil, bois de rose oil, grapefruit oil, lemon oil, mandarin oil, orange oil, oregano oil, lavender oil, Lindera oil, pine needle oil, pepper oil, rose oil, iris oil, sweet orange oil, tangerine oil, tea tree oil, tea seed oil, thyme oil, thymol oil, garlic oil, peppermint oil, onion oil, linaloe oil, Japanese mint oil, spearmint oil, linalool, limonene, geraniol, citral, eugenol and others as disclosed herein throughout. Preferably, the essential oil is a combination of two or more essential oils. More preferably the essential oils are selected from Melaleuca alternifolia oil, Lavandula officinalis oil, Cinnamomum zeylanicum oil, Eugenia caryophyllus oil, Cymbopogon flexuosus oil, Juniperus virginiana oil, Thymus vulgaris oil, Eugenia caryophyllus oil, Cordia curassavica oil, Varronia curassavica oil, Cordia Verbenacea oil, or combinations thereof.

In another embodiment, the formulation further comprises a fragrance.

In a third aspect, the present invention provides a method of reducing or inhibiting malodor in fabrics, textiles or clothing, comprising applying the formulation described above directly to a fabric, textile or clothing prior to use.

### DETAILED DESCRIPTION OF THE INVENTION

Methods and compositions comprising essential oils are provided herein for inhibiting or reducing textile unpleasant odors, which typically occurs in areas such as the axillae, feet, palms, back, neck, face, groin and other sweatier parts of the body. For the purposes of the present invention, the terms “composition” and “formulation” are used interchangeably.

Textile malodor is characterized by a sour, musty, sharp, oniony and/or fecal-like smell. The controlled release of essential oils that possess antibacterial and antiseptic properties leads to a bacteriostatic effect on clothes, therefore lowering the microbial load on clothes, and as such, the odor release through its enzymes is limited.

Textile malodor is generated due to bacterial biotransformation of sweat secretions. There are three major routes that lead to underarm/skin malodor. (1) Typical human, unusual, methyl-branched, odd-numbered long-chain fatty acids (LCFA) are degraded via β-oxidation into short-chain, volatile fatty acids (James et al. 2004, 2013). (2) Additionally, the release of short-chain fatty acids, such as E-3-methyl-2-hexenoic acid (3M2H), 3-hydroxy-3-methyl-hexanoic acid (HMHA), 3-hydroxy-3-methylhexanoic acid (3M3H), and a wide range of other structurally unusual VFAs, secreted as L-glutamine conjugates in apocrine glands, are considered as major components of the axillary malodor (Natsch et al. 2003, 2006; Zeng et al. 1991). After secretion by apocrine sweat glands, bacteria remove the L-glutamine residue with N^{α}-acyl-glutamine amino acylase and consequently releasing the VFAs. (3) Several thioalcohols, such as 3-methyl-3-sulfanyl-hexan-1-ol (3M3SH) and 2-methyl-3-sulphanylbutan-1-ol (2M3SB), as well as their isomers were also reported as important contributors to axillary malodor (Hasegawa, Kabuki, and Matsu Kane 2004; Natsch, Schmid, and Flashman 2004; M Trocar et al. 2004).

The present invention has three impacts. 1/ The present invention first relates to methods that limit the biochemical conversion of the precursor molecules into volatile and malodorous compounds. 2/ The present invention secondly relates to a natural perfuming impact through the release of natural and plant-derived fragrance molecules. And 3/ the present invention relates to the sustained and long-term release of bioactives on clothes, that are released upon contact with moisture, through friction with clothes, and by being degraded by local microbiome. As such, there is a long-term inhibitory impact on the microbiome, as well as long-term release of good fragrances.

In the context of the present invention, the terms textile or fabric may include fibers, natural (for example, cotton, silk, wool, and linen) or synthetic yarns spun from those fibers, and woven, knit, and non-woven fabrics made of those yarns. The scope of this invention includes fibers; and all synthetic fibers used in textile applications, including but not limited to synthetic cellulosic fibers (i.e., regenerated cellulose fibers such as rayon, and cellulose derivative fibers such as acetate fibers), regenerated protein fibers, acrylic fibers, polyolefin fibers, polyurethane fibers, and vinyl fibers, but excluding nylon and polyester fibers, and blends thereof. The term clothes or clothing as used herein may refer to any piece or item made of fabric, textile fur, leather and wool to be used or worn on the body.

According to the present invention, the biologically active compounds are at least one essential oil, which is a predominantly volatile material or materials isolated by some physical or chemical process from an odorous botanical source. It is apparent that essential oil components that are sufficiently active will also be useful in the practice of the invention.

In a preferred embodiment of the invention, the essential oil can be selected from the following non-limiting examples of sesame oil, pyrethrum, glycerol-derived lipids or glycerol fatty acid derivatives, cinnamon oil, cedar oil, clove oil, geranium oil, lemongrass oil, angelica oil, mint oil, turmeric oil, wintergreen oil, rosemary oil, anise oil, cardamom oil, caraway oil, chamomile oil, coriander oil, guaiac wood oil, cumin oil, dill oil, mint oil, parsley oil, basil oil, camphor oil, cananga oil, citronella oil, eucalyptus oil, fennel oil, ginger oil, copaiba balsam oil, perilla oil, cedarwood oil, jasmine oil, palmarosa sofia oil, western mint oil, star anise oil, tuberose oil, neroli oil, tolu balsam oil, patchouli oil, palmarosa oil, Chamaecyparis obtusa oil, Hiba oil, sandalwood oil, petitgrain oil, bay oil, vetiver oil, bergamot oil, Peru balsam oil, bois de rose oil, grapefruit oil, lemon oil, mandarin oil, orange oil, oregano oil, lavender oil, Lindera oil, pine needle oil, pepper oil, rose oil, iris oil, sweet orange oil, tangerine oil, tea tree oil, tea seed oil, thyme oil, thymol oil, garlic oil, peppermint oil, onion oil, linaloe oil, Japanese mint oil, spearmint oil, linalool, limonene, geraniol, citral, eugenol and others as disclosed herein throughout. Preferably, the essential oil is a combination of two or more essential oils. More preferably the essential oils are selected from Melaleuca alternifolia leaf oil, Lavandula officinalis flower oil, Cinnamomum zeylanicum bark oil, Eugenia caryophyllus flower oil, Cymbopogon flexuosus oil, Juniperus virginiana wood oil, Thymus vulgaris flower/leaf oil.

It is clear that a person skilled in the art is enabled to identify and select other plant genera or species that could be used as sources of essential oils or individual components thereof suitable for the present invention, without departing from the spirit and scope of the present invention. Also, the skilled in the art is enabled to determine the components and concentrations thereof in a chosen essential oil, for example, via techniques including, but not limited to, HPLC, GC, GC-MS, GC-MS-MS, LC-MS, LC-NMR, LC-DAD, LC-MS-MS. Thus, the skilled in the art may determine the amount of essential oil to be used in the manufacture of the formulation for inhibiting or reducing textile malodor, in order to result in appropriate mixture and amounts in said formulation.

The formulation may further include more than one essential oil, providing a synergistic effect. The term “synergistic effect” is defined as the effect achieved when two or more essential oils work together to produce a result not obtainable by any of the actives independently. In this sense, the combination of two or more essential oils may result in an enhanced malodor-reducing efficacy. It is clear that a skilled person is enabled to select other combinations that may result in a synergistic effect in the malodor inhibition or reduction, without departing from the spirit and scope of the invention.

A preferred embodiment comprises the use of a controlled release technology (CRT) to obtain an extended, controlled, and long-lasting delivery of the compounds (Kumari, Yadav, and Yadav 2010; Montalvo-Ortiz, Sosa, and Griebenow 2012). Encapsulation of natural and biological agents is beneficial to the cosmetics, washing and cleaning sectors to preserve the sensory characteristics of fragrance molecules (Ammala 2013). Encapsulation may also prevent evaporation at the manufacturing site. Encapsulation can help to reduce the frequency of perfume dermatitis (Lee et al. 2002), and it helps to have a controlled and long-term delivery of the actives on site. In summary, the CRT prevents early evaporation, enhanced stability of the ingredients, and an effective delivery of the active agents over a prolonged and sustained time period.

Among the several available CRT technologies, the present invention uses preferably microparticles or nanoparticles for the delivery of essential oils. Nanoparticles were first developed around 1970 for carrying vaccines and anticancer drugs. Besides controlling the release of drug molecules, these carriers also protect essential oils against possible thermal or photo degradation which assures increased stability and function, consequently extending the final product shelf life. Considering these features, these systems can represent an interesting approach for overcoming essential oils limitations. Micro/Nanocarriers can potentially protect the essential oils from oxidation or evaporation, as well as facilitate their antimicrobial activity by providing diverse diffusion properties through biological membranes due to the particles dimension (São Pedro, A. et al. 2013).

Also according to the present invention, the essential oils are present in the formulation solubilized, in the form of a suspension or in form of microparticles and/or nanoparticles. Preferably, the essential oils are present in the formulation in the form of microparticles or nanoparticles. According to the present invention, the nanocarriers can be structured by a great variety of material and designs. Preferably, the nanocarrier systems of the present invention, are lipid-based particles, nano emulsions and biocompatible polymer-based particles. The essential oils can also be associated with inorganic nanocarriers such as metal oxide base particles and nano clay. More preferably, the essential oils of the present invention are present in the form of liposomes, solid lipid nanoparticles, polymeric nanospheres, polymeric nano capsules, or nano emulsions.

Polymeric nanoparticles may be classified as nano capsules and nanospheres. Nano capsules have two compartments; a polymeric wall and a core, which is commonly oily. Nanospheres are matrix systems. In these systems, the essential oil may be conjugated with the polymeric matrix or wall or it may be in the oily core. Optionally, these particles are prepared by different techniques as, e.g., nanoprecipitation or solvent displacement. Optionally, the nanoparticles of the present invention may be selected from any commercially available essential oil-containing nanoparticles.

The product formulation may vary depending on what is appropriate or desired for the form. Such formulation may be available in a variety of product forms, for example, as aerosols, pump and squeeze sprays, powders, lotions, emulsions, gels, creams.

It also may be anhydrous, or water based, utilizing such combination of components as to provide the desired profile of dose and aesthetics. In general, it will include a vehicle/carrier, dispersant, emollient, fragrance, surfactant and structurants. Besides the use in the manufacture of products for inhibition or reduction of malodor in clothes, the essential oils may also be incorporated into a reservoir or other type of “patch” or plaster or embedded in a matrix for controlled release in other sweaty parts of the body, for example, but not limited to, sole insert to deliver to foot, or sock or glove to deliver to sole/palm. Also, the essential oils may be incorporated into cosmetic or pharmaceutical products, e.g., moisturizing creams and body sprays.

The formulation may comprise a vehicle, and at least one essential oil, wherein the total amount of essential oils in the composition ranges from 0.01% to 40% by weight relative to the total weight of the composition (w/w), with a preferred range of 1% to 35%, more preferred ranges of 1% to 20%, 1% to 10% and the most preferred range of 1% to 5%. Preferably, the composition comprises at least two essential oils, at least three essential oils, at least four essential oils. More preferably, the composition comprises at least six essential oils, and most preferably, the composition comprises at least eight essential oils. In one embodiment, the essential oils in the composition are present in different amounts of % weight relative to one another. In another, more preferred embodiment, the essential oils in the composition are present in the same amounts of % weight relative to one another.

It is evident that the skilled in the art is enabled to determine the more appropriate w/w percentage of essential oil to be included in a formulation for inhibiting or reducing textile malodors, according to scientific studies and/or regulation guidelines, to guarantee the best effect together with the product safety. The vehicle can be inert or can possess cosmetic, physiological and/or pharmaceutical benefits on its own. Vehicles may comprise water, alcohol, an anhydrous solution or a hydrophobic matrix and can be formulated with non-essential additional ingredients such as liquid or solid emollients, enhancers or modifiers, structurants, solvents, thickeners, surfactants, co-surfactants, fragrances, sensory modifiers, emulsifiers, gelling agents, dispersants, deodorant, suspending agents, wash-off agents, controlled release agents, anti-fungal, anti-bacterial, UV-protection, etc., or other activities which are intended to impart benefit to the textile or skin itself.

Other fragrances, when present, typically comprise up to about 1% of the total product. Coloring agents and preservatives can be added as desired. Propellants commonly employable in aerosol compositions herein comprise hydrocarbons (or much less desirably halohydrocarbons) having a boiling point of below 10° C. and especially those with a boiling point below 0° C. It is especially preferred to employ liquefied hydrocarbon gases, and especially C3 to C6 hydrocarbons, including propane, isopropane, butane, isobutane, pentane and isopentane and mixtures of two or more thereof. Preferred propellants are isobutane, isobutane/isopropane, isobutane/propane and mixtures of isopropane, isobutane and butane. Other propellants that can be contemplated include alkyl ethers, such as dimethyl ether or compressed non-reactive gasses such air, nitrogen or carbon dioxide.

In another aspect, the invention provides a method for reducing or inhibiting malodors in fabrics, textiles or clothing, comprising applying the formulation of the invention on the fabrics, textiles or clothing prior to use by the subject, and let the composition dry on the fabrics, textiles or clothing.

Many alterations and modification may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

### EXAMPLES

### Example 1 - Nanoparticles

The essential oils nanoparticles were formulated in a composition suitable to be used as a spray as follows (Formulation 1):
Nanoparticles comprising:
   Cymbopogon flexuosus oil
   Eugenia caryophyllus flower oil
   Eugenia caryophyllus leaf oil
   Thymus vulgaris flower/leaf oil
   Melaleuca alternifolia leaf oil
   Juniperus virginiana wood oil
   Lavandula officinalis flower oil
   Cinnamomum zeylanicum bark oil
Aqua; and
C12-14 pareth-3, Caprylyl glycol, Phenoxyethanol, Quaternium-22, Cetearyl alcohol, Linoleic acid, Oleic acid, Poloxamer 407, Polysorbate 80, Polyvinyl alcohol, Ppg-15 stearyl ether, Sodium bicarbonate, Steareth-2, Steareth-21, Stearic acid, Propylene glycol as additives/components of the nanocapsules.

The nanoparticles were prepared by and purchased from Nanovetores Tecnologia S.A. (Santa Catarina, Brazil).

### Characterization of the Nanoparticles

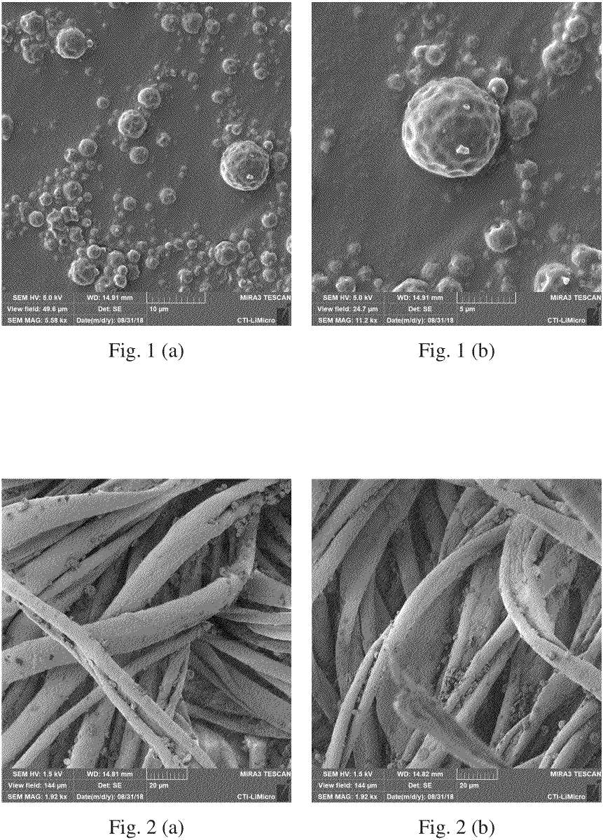

The morphology and size of the micro-capsules was evaluated using a MIRA3 TESCAN scanning electron microscopy (SEM - TESCAN Mira3 XMU, USA Inc.) operated between 1.5 and 5 kV. Samples were prepared for SEM by air drying of 10 µL of the resulting suspension on Formvar-coated copper grids. Pictures were taken using a Nikon Ti wide field fluorescence microscope (Nikon, Japan) at the excitation wavelength at 488 nm (FIG. 1). Size distributions of the micro-capsules were determined using a dynamic light scattering (DLS) (Malvern PCS-100-M Instruments, Malvern, UK) equipped with a He-Ne laser, operating at 633 nm at 28° C. and a fixed scattering angle of 150°. The stability of the capsules was assessed by measuring particle size distribution for seven days. Size distribution calculation was based on measuring over 100 micro-capsules. The interaction of the micro-capsules was also evaluated directly on textile fibers. Uncolored cotton fibers were inoculated with the micro-capsules and evaluated using scanning electron microscopy, as done before (FIG. 2).

The size of the nanoparticles ranged between 200 nm and 1100 nm. The median particle size of the microcapsules was 572 nm and the average particle size was 695.5±286.6 nm. Pictures of the nanoparticles were taken in both aqueous solution and on textile. The nanoparticles have a clear round shape (FIG. 1). The nanoparticles attached clearly to the cotton fibers (FIG. 2).

### Example 2 - In Vitro Experiments

### Nutrient Broth

Textile bacteria were cultured in liquid medium using nutrient broth. To compose 500 mL nutrient broth, 6.5 g nutrient broth without agar (Oxoid LTD, Basingstoke, Hampshire, England) was added to a Schott bottle of 500 mL. The bottle was diluted with distilled water until 500 mL. Afterwards, the bottle was shaken and autoclaved. Finally, the bottle was distributed over 10 mL tubes in a sterile manner. The tubes were stored in a cold room (ca. 4° C.) to make sure they remain sterile.

### PBS-Solution

To dilute bacterial samples, a phosphate-buffered saline solution (PBS) was prepared. One needed to put one tablet of 2 g in 200 mL of distilled water in order to receive a 0.01 M phosphate buffer, 0.0027 M potassium chloride and 0.137 M sodium chloride with an overall pH of 7.4 at 25° C. After sterilization, the solution was ready for use.

### Physiological Water

Physiological water was used to serially dilute bacterial cultures. The physiological solution contained 4.25 g NaCl (Carl Roth GmbH, Karlsruhe, Germany) for 500 mL of sterile distilled water. Before use, the solution was sterilized at 121° C. for 30 min.

### Bacterial Cultures

Mixed or pure bacteria were obtained from the internal culture collection, stored in a -80° C. freezer with the cryoprotectant glycerol. Selected microorganism(s) may be isolated from a natural environment (e.g., the worn clothes of a person) or purchased from a suitable commercial source such as the American Type Culture Collection (ATCC) (10801 University Boulevard, Manassas, VA 20110 USA).

Bacteria were grown until plateau phase before conducting in vitro tests. A bacterium, stored in the -80° C. freezer, was defrosted and spread with a Drigalski spatula on a nutrient agar plate. This plate was incubated for 24 hours at 37° C. Afterwards, a colony was picked up from the medium and brought to a new nutrient agar plate by using the streak plate method. After 24 h of incubation, a pure strain was brought into a 10 mL tube with sterile nutrient broth. After 24 h, the bacterium was ready for use. Growth curves of each selected strain was performed to check for exponential, plateau and decay phase. This was done measuring the maximal optical density (ODₘₐₓ) using a spectrophotometer.

### AATCC 100 Method 100-1999

The AATCC 100 method 100-1999 was performed, which is widely used test to verify antibacterial finishing agents in textile materials. To quantitatively analyze the degree of antibacterial activity in textile materials, a test was performed which consisted of exposure of the samples to two representative microorganisms: Staphylococcus aureus (Gram-positive) ATCC 6538 and Escherichia coli (Gram-negative) ATCC 43893 serotype O:124. Antibacterial activity was evaluated by counting the colony forming units (CFU) on 1 cm² of cotton fibers, which were inoculated at time zero with 1 mL of the bacterial suspensions, at a concentration of 1.5 × 108 CFU /mL and incubated at 37° C. for 24 hours. After incubation, the bacteria are removed by washing with a sterile PBS buffer, and the number of bacteria present is then determined. Serial dilutions were performed in PBS buffer and plating was performed on nutrient agar. After incubation at 37° C. for 24 hours, CFU counts were performed and the reduction was calculated percentage by comparing to the amount of CFU at time zero.

### Minimally Inhibitory Concentration (MIC) Determination

The minimum inhibitory concentration (MIC) of the nanoparticles, dissolved in a water solution, was verified in an aqueous solution. This was verified in two different manners: 1/ by measuring the optical density (OD) at a wavelength of 610 nm and 2/ by measuring the plate count on nutrient agar. Clothing bacteria, obtained from a worn shirt, containing Staphylococcus, Corynebacterium, Micrococcus, Acinetobacter spp. were harvested using a cotton tip and dissolved in sterile saline water. The bacteria were grown on a nutrient agar plate and dissolved in nutrient broth and grown to a stationary phase. Optical density measurements were performed at 0 h. Plate counts, and as such, bacterial viability, were performed at 0 h.

Serial dilutions of the nanoparticle solution, in a concentration of 0 to 200 µg/mL, were dissolved in nutrient broth containing a dense concentration of textile bacteria. The nutrient broth tubes were incubated at 37° C. for 24 h while shaking.

OD measurements were conducted at 24 h. Plate counts were measured at 24 h. The difference in optical density and plate counts between 0 h and 24 h was calculated. Growth or inhibition was determined, based on the OD measurements (Table 3) and plate counts (Table 4). The minimal concentration for inhibition of textile bacteria was determined when a negative growth was found.

The AATCC method indicated a strong inhibition of representative Gram-positive and Gram-negative bacteria on textiles. Inhibition of bacteria was achieved at a concentration of 0.1 and 0.01 mL per cm2 of textile (Table 1) (Table 2).

TABLE 1

AATCC 100 results for Staphylococcus aureus (ATCC 6538)

| Bacterium: S. aureus | Sample n° | CFU/mL | Average | Growth | Inhibition percentage |
|---|---|---|---|---|---|
| Control | 16 31 12 | 8.0E+06 1.5E+07 6.0E+06 | 9.67E+06 | 100% | 0% |
| Fabric + 0.1 mL Formulation 1 | 7 8 13 | 3.5E+05 4.0E+05 6.5E+05 | 4.67E+05 | 5% | 95% |
| Fabric + 0.01 mL Formulation 1 | 11 36 16 | 5.5E+05 1.8E+06 8.0E+05 | 1.03E+06 | 11% | 89% |

Clear inhibition was observed at both 0.01 mL and 0.1 mL spray concentration.

TABLE 2

AATCC 100 results for Escherichia coli (ATCC 43893)

| Bacterium: E. coli | Sample n° | CFU/mL | Average | Growth | Inhibition percentage |
|---|---|---|---|---|---|
| Control | 35 25 28 | 1.8E+07 1.3E+07 1.4E+07 | 1.47E+07 | 100% | 0% |
| Fabric + 0.1 mL Formulation 1 | 3 4 11 | 1.5E+06 2.0E+06 5.5E+06 | 3.00E+06 | 38% | 62% |
| Fabric + 0.01 mL Formulation 1 | 11 9 13 | 5.5E+06 4.5E+06 6.5E+06 | 5.50E+06 | 21% | 79% |

Clear inhibition was observed at both 0.01 mL and 0.1 mL spray concentration.

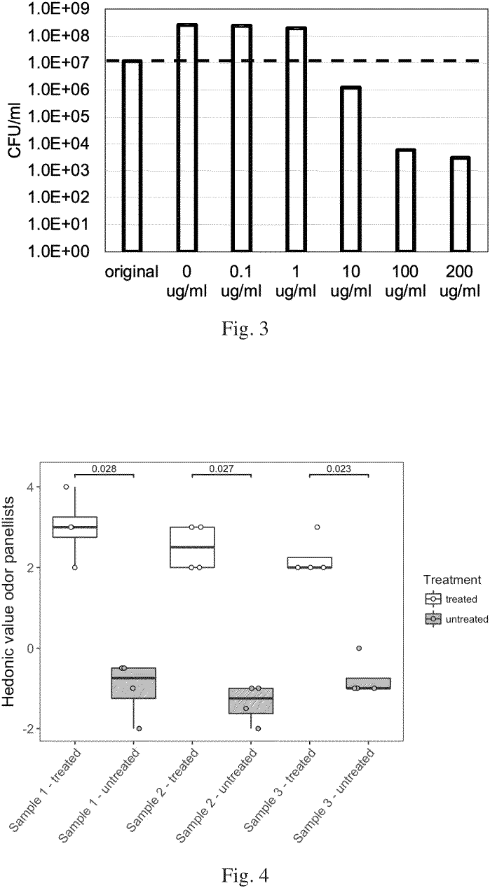

The minimum inhibitory concentration (MIC) of the nanoparticles in aqueous solution was found at 10 µg/mL. Both the optical density (Table 3) and the plate count (Table 4) measurements found inhibition of textile bacteria in aqueous solution at 10 µg/mL (FIG. 3).

TABLE 3

Minimal inhibitory concentration (MIC) determination based on OD measurements

| OD 24 | | | | OD | |
|---|---|---|---|---|---|
| µg/mL | NPs | OD 0 h | h | difference | growth/inhibition |
| 0 | 0 µL | 0.093 | 0.154 | 0.061 | growth |
| 0.1 | 1 µL | 0.114 | 0.160 | 0.046 | growth |
| 1 | 10 µL | 0.171 | 0.204 | 0.033 | growth |
| 10 | 100 µL | 0.812 | 0.738 | -0.074 | inhibition |
| 100 | 1 mL | 2.805 | 2.391 | -0.414 | inhibition |
| 200 | 2 mL | 3.181 | 2.769 | -0.412 | inhibition |

TABLE 4

Minimal inhibitory concentration (MIC) determination based on plate count measurements

| µg/mL | NPs | CFU/mL 0 h | CFU/mL 24 h | OD difference | log difference | growth/inhibition |
|---|---|---|---|---|---|---|
| 0 | 0 µL | 1.2E+07 | 2.7E+08 | 2.5E+08 | +1 | growth |
| 0.1 | 1 µL | 1.2E+07 | 2.4E+08 | 2.3E+08 | +1 | growth |
| 1 | 10 µL | 1.2E+07 | 1.9E+08 | 1.8E+08 | +1 | growth |
| 10 | 100 µL | 1.2E+07 | 1.3E+06 | -1.0E+07 | -1 | inhibition |
| 100 | 1 mL | 1.2E+07 | 6.0E+03 | -1.2E+07 | -4 | inhibition |
| 200 | 2 mL | 1.2E+07 | 3.0E+03 | -1.2E+07 | -4 | inhibition |

### Example 3 - In Vivo Experiments

### Study Design

Healthy volunteers were recruited to test the present invention. Volunteers were instructed to wear clothes during normal workdays. Clothes were treated on one underarm region with an aqueous solution of nanoparticles, while the other side was not treated and served as control. Samples were taken and verified at 24 h and 48 h. (Volunteers were asked to do self-assessment of odor of the worn clothes, on both sides.) The worn clothes were subjected to objective hedonic assessment by an odor panel. Both sides were assessed by the odor panel.

Clothes were inoculated with bacterial cultures after which nano capsules in different concentrations were added, for MIC determination. Pictures were taken of the treated clothes using TEM. Using serial dilutions, plate counts of the treated samples were determined.

### Odor Assessment

The odor panel was trained and selected, and samples were rated as previously described (C. Callewaert et al. 2013). Odor measurement was performed by means direct assessment of the used textile. The shirts were presented in an anonymized manner to a panel of four selected and screened human assessors (two men, two women). Assessors were selected by means of sensitivity to dilutions of n-butanol and wastewater, and by means of the triangle test (Amoore, Venstrom, and Nutting 1972). In the triangle test, each member of the panel was presented three flasks, two of which were the same but the third contained a different odor. The flask was shaken, the stopper was removed, after which the vapors were evaluated. The panelists had to correctly identify the flask with the different odor. In the dilution test, each member of the panel was presented six flasks with increasing concentration of n-butanol and wastewater, starting with a flask without addition of n-butanol or wastewater. The panelists had to correctly place the flasks according concentration. The triangle and dilution test were repeated three times, with a minimum of two days in between each measurement. Assessors with minimum 85% correct answers were selected for the panel. A representative panel was recruited from a pool of the 20 people. Training of the assessors was conducted through odor references (ammonia, cheese, axillary sample) and experience. The references were assessed in group to compromise hedonic value and intensity. The room in which the tests were conducted, was free from extraneous odor stimuli e.g. caused by smoking, eating, soaps, perfume, etc. The odor assessors were familiar with the olfactometric procedures and met the following conditions: (i) older than 16 years and willing to follow the instructions; (ii) no smoking, eating, drinking (except water) or using chewing gum or sweets 30 min before olfactometric measurement; (iii) free from colds, allergies or other infections; (iv) no interference by perfumes, deodorants, body lotions, cosmetics or personal body odor; (v) no communication during odor assessment. The samples were assessed by the following odor characteristics: hedonic value and intensity, as well as description of the odor. The hedonic value indicates the pleasantness of the odor and varies from -8 (very unpleasant over 0 (neutral) to +8 (very pleasant). The intensity indicates the quantity of the odor and varies between 0 (no odor) to 10 (very strong / intolerable). Odor characteristics were noted when clearly present. Participants also did a self-evaluation of the odor.

### Bacterial Survival Assessment

A mixed culture of skin bacteria was dissolved in nutrient broth, after which the CFU count was determined of the originally added bacterial culture. The bacterial culture was used to inoculate textiles of nutrient broth with or without microcapsules. Samples were taken of the treated clothes or broth using sterile swabs, pre-moistened in sterile physiological water. The samples were serially diluted in sterile physiological water, for 7 logs. 100 µl of the serially diluted samples were plated on nutrient agar plates and incubated at 37° C. for 24 h. The grown colonies were counted to determine the plate counts after 24 h.

### Spray Experiment With Micro-Encapsulated Actives

Healthy volunteers were recruited that applied the micro-encapsulated actives in a spray formula, as described in Formula 1, on the clothes. The volunteers applied the micro-encapsulated actives in the underarm region of the clothing, that was worn directly on skin.

A concentration of 0.1 mL/cm² textile was applied. One underarm region (left side) was untreated, while the other underarm region of the textile (right side) was treated with the nanoparticles. The volunteer applied the spray on a daily basis. The first application was when a new shirt was worn, at 0 h. The second application was the day after, at 24 h, when the same shirt was worn for the second day. The underarm region of the clothes was sampled at 12 h and at 36 h. It was tested and verified on agar plate that the spray containing the micro-encapsulated actives was sterile and did not contain any microorganisms.

Overall, participants reported a decreased clothing odor in the underarm clothing region (self-assessment).

The independent odor panel also found better hedonic values when the nanoparticles were applied (FIG. 4). The results were obtained after 24 h of wearing by participants. One underarm region of the textile was treated, while the other side remained untreated. The textiles were wool (Sample 1) and cotton (Sample 2 and 3). The cotton shirts were worn directly on the skin, while the wool shirt was worn on a shirt underneath. The odor panel indicated that the treated side smelled like “clove, wood, essential oils, and fresh”, while the untreated side smelled like “slight sour, bit musty, and used”. Significant differences were obtained: the treated side smelled significantly more pleasant (p=0.028, Mann-Whitney U-test for Sample 1 - wool shirt), (p=0.027, Mann-Whitney U-test for Sample 2 - cotton shirt) and (p=0.023, Mann-Whitney U-test for Sample 3 - cotton shirt).

The bacterial load on clothes did not exceed 10_7 CFU/cm². A bacteriostatic effect was noticed on the applied textiles.

### REFERENCES

Adukwu, Emmanuel C, Melissa Bowles, Valerie Edwards-Jones, and Heather Bone. 2016. “Antimicrobial Activity, Cytotoxicity and Chemical Analysis of Lemongrass Essential Oil (Cymbopogon Flexuosus) and Pure Citral.” Applied Microbiology and Biotechnology 100(22): 9619-27. https://doi.org/10.1007/s00253-016-7807-y.

Ammala, Anne. 2013. “Biodegradable Polymers as Encapsulation Materials for Cosmetics and Personal Care Markets.” International journal of cosmetic science 35(2): 113-24.

Amoore, J E, D Venstrom, and M D Nutting. 1972. “Sweaty Odor in Fatty Acids - Measurements of Similarity, Confusion and Fatigue.” Journal of Food Science 37(1): 33-35.

Boonme, Prapaporn, and Sarunyoo Songkro. 2010. “Antiperspirants and Deodorants: Active Ingredients And.” (01): 5-10.

Bouslimani, Amina et al. 2019. “The Impact of Skin Care Products on Skin Chemistry and Microbiome Dynamics.” BMC Biology 17(1): 47. https://doi.org/10.1186/s12915-019-0660-6.

Callewaert, C., E. De Maeseneire, T. Van de Wiele, and N. Boon. 2013. “Bacterial and Odor Profile of Polyester and Cotton Clothes after a Fitness Session.” Communications in agricultural and applied biological sciences 78(1).

Callewaert, Chris, Evelyn De Maeseneire, et al. 2014. “Microbial Odor Profile of Polyester and Cotton Clothes after a Fitness Session.” Applied and Environmental Microbiology 80(21): 6611-19.

Callewaert, Chris, Prawira Hutapea, Tom Van de Wiele, and Nico Boon. 2014. “Deodorants and Antiperspirants Affect the Axillary Bacterial Community.” Archives of Dermatological Research 306(8): 701-10.

Carson, C F, K A Hammer, and T V Riley. 2006. “Melaleuca Alternifolia (Tea Tree) Oil: A Review of Antimicrobial and Other Medicinal Properties.” Clinical microbiology reviews 19(1): 50-62. https://www.ncbi.nlm.nih.gov/pubmed/16418522.

Dravniek, A, B Krotoszy, W E Lieb, and E Jungerma. 1968. “Influence of an Antibacterial Soap on Various Effluents from Axillae.” Journal of the Society of Cosmetic Chemists 19(9): 611-26.

Famuyide IM, Aro AO, Fasina FO, Eloff JN, McGaw LJ. 2019. “Antibacterial and antibiofilm activity of acetone leaf extracts of nine under-investigated south African Eugenia and Syzygium (Myrtaceae) species and their selectivity indices.” BMC Complement Altern Med. 19(1):141. Published 2019 Jun 20. doi:10.1186/s12906-019-2547-z

Fani, Mohammadmehdi, and Jamshid Kohanteb. 2017. “In Vitro Antimicrobial Activity of Thymus vulgaris Essential Oil Against Major Oral Pathogens.” Journal of evidence-based complementary & alternative medicine vol. 22,(4): 660-666. doi:10.1177/2156587217700772

Gavanji S, Mohammadi E, Larki B, Bakhtari A. 2014. “Antimicrobial and cytotoxic evaluation of some herbal essential oils in comparison with common antibiotics in bioassay condition.” Integr Med Res. 3(3):142-152. doi:10.1016/j.imr.2014.07.001

Hasegawa, Y, M Yabuki, and M Matsukane. 2004. “Identification of New Odoriferous Compounds in Human Axillary Sweat.” Chemistry & Biodiversity 1(12): 2042-50.

James, A G et al. 2013. “Microbiological and Biochemical Origins of Human Axillary Odor.” FEMS Microbiology Ecology 83(3): 527-40.

James, A G, J Casey, D Hyliands, and G Mycock. 2004. “Fatty Acid Metabolism by Cutaneous Bacteria and Its Role in Axillary Malodor.” World Journal of Microbiology & Biotechnology 20(8): 787-93.

Jirovetz, Leopold et al. 2006. “Comparative Study on the Antimicrobial Activities of Different Sandalwood Essential Oils of Various Origin.” Flavour and Fragrance Journal 21(3): 465-68. https://doi.org/10.1002/ffj.1625.

Kumari, Avnesh, Sudesh Kumar Yadav, and Subhash C Yadav. 2010. “Biodegradable Polymeric Nanoparticles Based Drug Delivery Systems.” Colloids and Surfaces B: Biointerfaces 75(1): 1-18. http://www.sciencedirect.com/science/article/pii/S0927776509004111.

Lee, H Y, S J Lee, I W Cheong, and J H Kim. 2002. “Microencapsulation of Fragrant Oil via in Situ Polymerization: Effects of PH and Melamine-Formaldehyde Molar Ratio.” Journal of microencapsulation 19(5): 559-69.

Makin, Stephen A., and Michael R. Lowry. 1999. “Deodorant Ingredients.” In K Laden, Editor. Antiperspirants and Deodorants, New York: Marcel Dekker, 169-214.

McQueen, R H, R M Laing, H J L Brooks, and B E Niven. 2007. “Odor Intensity in Apparel Fabrics and the Link with Bacterial Populations.” Textile Research Journal 77(7): 449-56.

Montalvo-Ortiz, Brenda L, Brian Sosa, and Kai Griebenow. 2012. “Improved Enzyme Activity and Stability in Polymer Microspheres by Encapsulation of Protein Nanospheres.” AAPS PharmSciTech 13(2): 632-36. https://doi.org/10.1208/s12249-012-9794-3.

N′dédianhoua, K Soro, Karima Majdouli, Youssef Khabbal, and Touryia Zaïr. 2014. “Chemical Composition and Antibacterial Activity of Lavandula Species L. Dentata L., L. Pedunculata Mill and Lavandula Abrialis Essential Oils from Morocco against Food-Borne and Nosocomial Pathogens.” International Journal of Innovation and Applied Studies 7(2): 774.

Natsch, A et al. 2003. “A Specific Bacterial Aminoacylase Cleaves Odorant Precursors Secreted in the Human Axilla.” Journal of Biological Chemistry 278(8): 5718-27.

Natsch, A, S Derrer, F Flachsmann, and J Schmid. 2006. “A Broad Diversity of Volatile Carboxylic Acids, Released by a Bacterial Aminoacylase from Axilla Secretions, as Candidate Molecules for the Determination of Human-Body Odor Type.” Chemistry & Biodiversity 3(1): 1-20.

Natsch, A, J Schmid, and F Flachsmann. 2004. “Identification of Odoriferous Sulfanylalkanols in Human Axilla Secretions and Their Formation through Cleavage of Cysteine Precursors by a C-S Lyase Isolated from Axilla Bacteria.” Chemistry & Biodiversity 1(7): 1058-72.

O’Bryan, Corliss A, Sean J Pendleton, Philip G Crandall, and Steven C Ricke. 2015. “Potential of Plant Essential Oils and Their Components in Animal Agriculture - in Vitro Studies on Antibacterial Mode of Action.” Frontiers in veterinary science 2: 35.

Pandima Devi, K. Arif Nisha, S. Sakthivel, R. Karutha Pandian S. 2010. “Eugenol (an essential oil of clove) acts as an antibacterial agent against by disrupting the cellular membrane.” Journal of Ethnopharmacology, 130-1: 107-115, https://doi.org/10.1016/j.jep.2010.04.025.

Pina□Vaz, C., Gonçalves Rodrigues, A., Pinto, E., Costa□de□ Oliveira, S., Tavares, C., Salgueiro, L., Cavaleiro, C., Gonçalves, M. and Martinez□ de□Oliveira, .J. 2004. “Antifungal activity of Thymus oils and their major compounds”. Journal of the European Academy of Dermatology and Venereology, 18: 73-78. doi:10.1111/j.1468-3083.2004.00886.x

Prabuseenivasan, Seenivasan, Manickkam Jayakumar, and Savarimuthu Ignacimuthu. 2006. “In Vitro Antibacterial Activity of Some Plant Essential Oils.” BMC complementary and alternative medicine 6: 39.

Rajkumar, V, and J S Berwal. 2003. “Inhibitory Effect of Clove (Eugenia Caryophyllus) on Toxigenic Molds.” JOURNAL OF FOOD SCIENCE AND TECHNOLOGY-MYSORE- 40(4): 416-18.

Ranasinghe, P., Pigera, S., Premakumara, G.S. et al. 2013. “Medicinal properties of ‘true’ cinnamon (Cinnamomum zeylanicum): a systematic review.” BMC Complement Altern Med 13, 275 doi:10.1186/1472-6882-13-275

de Rapper S, Kamatou G, Viljoen A, van Vuuren S. 2013. “The In Vitro Antimicrobial Activity of Lavandula angustifolia Essential Oil in Combination with Other Aroma-Therapeutic Oils.” Evid Based Complement Alternat Med. 2013:852049. doi:10.1155/2013/852049

Rota, María C et al. 2008. “Antimicrobial Activity and Chemical Composition of Thymus Vulgaris, Thymus Zygis and Thymus Hyemalis Essential Oils.” Food Control 19(7): 681-87. http://www.sciencedirect.com/science/article/pii/S095671350700151X.

Safaei-Ghomi J, Ahd AA. 2010. Antimicrobial and antifungal properties of the essential oil and methanol extracts of Eucalyptus largiflorens and Eucalyptus intertexta. Pharmacogn Mag. 6(23):172-175. doi:10.4103/0973-1296.66930

São Pedro, André & Santo, Islane & Detoni, Cassia & Silva, Cinara & Cabral-Albuquerque, Elaine. 2013. “The use of nanotechnology as an approach for essential oil-based formulations with antimicrobial activity.”

Schelz, Zsuzsanna, Joseph Molnar, and Judit Hohmann. 2006. “Antimicrobial and Antiplasmid Activities of Essential Oils.” Fitoterapia 77(4): 279-85.

Shafaghat, Ali, Farshid Salimi, and Vahid Amani-Hooshyar. 2012. “Phytochemical and Antimicrobial Activities of Lavandula Officinalis Leaves and Stems against Some Pathogenic Microorganisms.” Journal of Medicinal Plants Research 6(3): 455-60.

Shelley, W B, H J Hurley, and A C Nicholas. 1953. “Axillary Odor: Experimental Study of the Role of Bacteria, Apocrine Sweat, and Deodorants.” Archives of Dermatology and Syphilology 68: 430-46.

Sienkiewicz, Monika et al. 2014. “The Biological Activities of Cinnamon, Geranium and Lavender Essential Oils.” Molecules (Basel, Switzerland) 19(12): 20929-40.

Szostak-Kotowa, J. 2004. “Biodeterioration of Textiles.” International Biodeterioration & Biodegradation 53: 165-70.

Troccaz, M et al. 2004. “3-Methyl-3-Sulfanylhexan-1-Ol as a Major Descriptor for the Human Axilla-Sweat Odor Profile.” Chemistry & Biodiversity 1(7): 1022-35.

Troccaz, Myriam et al. 2015. “Mapping Axillary Microbiota Responsible for Body Odors Using a Culture-Independent Approach.” Microbiome 3(1): 1-15. http://dx.doi.org/10.1186/s40168-014-0064-3.

Unlu, Mehmet et al. 2010. “Composition, Antimicrobial Activity and in Vitro Cytotoxicity of Essential Oil from Cinnamomum Zeylanicum Blume (Lauraceae).” Food and Chemical Toxicology 48(11): 3274-80. http://www.sciencedirect.com/science/article/pii/S0278691510005648.

Yang, Xian, Xue Zhang, Shui-Ping Yang, and Wei-Qi Liu. 2013. “Evaluation of the Antibacterial Activity of Patchouli Oil.” Iranian journal of pharmaceutical research : IJPR 12(3): 307-16. https://www.ncbi.nlm.nih.gov/pubmed/24250637.

Zeng, X N et al. 1991. “Analysis of Characteristic Odors from Human Male Axillae.” Journal of Chemical Ecology 17(7): 1469-92.

1. Use of a combination of two or more essential oils, selected from Melaleuca alternifolia oil, Lavandula officinalis oil, Cinnamomum zeylanicum oil, Eugenia caryophyllus oil, Cymbopogon flexuosus oil, Juniperus virginiana oil, Thymus vulgaris oil for the manufacture of a composition to prevent malodor in fabrics, textiles or clothing, wherein the two or more essential oils are contained in nano capsules.

2. Use according to claim 1, wherein the composition may also comprise sesame oil, pyrethrum, cinnamon oil, cedar oil, clove oil, geranium oil, lemongrass oil, angelica oil, mint oil, turmeric oil, wintergreen oil, rosemary oil, anise oil, cardamom oil, caraway oil, chamomile oil, coriander oil, guaiac wood oil, cumin oil, dill oil, mint oil, parsley oil, basil oil, camphor oil, cananga oil, citronella oil, eucalyptus oil, fennel oil, ginger oil, copaiba balsam oil, perilla oil, cedarwood oil, jasmine oil, palmarosa sofia oil, western mint oil, star anis oil, tuberose oil, neroli oil, tolu balsam oil, patchouli oil, palmarosa oil, Chamaecyparis obtusa oil, Hiba oil, sandalwood oil, petitgrain oil, bay oil, vetivert oil, bergamot oil, Peru balsam oil, bois de rose oil, grapefruit oil, lemon oil, mandarin oil, orange oil, oregano oil, lavender oil, Lindera oil, pine needle oil, pepper oil, rose oil, iris oil, sweet orange oil, tangerine oil, tea tree oil, tea seed oil, thyme oil, thymol oil, garlic oil, peppermint oil, onion oil, linaloe oil, Japanese mint oil, spearmint oil, linalool, limonene, geraniol, citral, or eugenol.

3. Formulation comprising a combination of two or more essential oils, selected from Melaleuca alternifolia oil, Lavandula officinalis oil, Cinnamomum zeylanicum oil, Eugenia caryophyllus oil, Cymbopogon flexuosus oil, Juniperus virginiana oil, Thymus vulgaris oil; and a vehicle for use in preventing malodor in fabrics, textiles or clothing, wherein the two or more essential oils are contained in nano capsules.

4. Formulation according to claim 3, wherein the composition may also comprise sesame oil, pyrethrum, cinnamon oil, cedar oil, clove oil, geranium oil, lemongrass oil, angelica oil, mint oil, turmeric oil, wintergreen oil, rosemary oil, anise oil, cardamom oil, caraway oil, chamomile oil, coriander oil, guaiac wood oil, cumin oil, dill oil, mint oil, parsley oil, basil oil, camphor oil, cananga oil, citronella oil, eucalyptus oil, fennel oil, ginger oil, copaiba balsam oil, perilla oil, cedarwood oil, jasmine oil, palmarosa sofia oil, western mint oil, star anise oil, tuberose oil, neroli oil, tolu balsam oil, patchouli oil, palmarosa oil, Chamaecyparis obtusa oil, Hiba oil, sandalwood oil, petitgrain oil, bay oil, vetiver oil, bergamot oil, Peru balsam oil, bois de rose oil, grapefruit oil, lemon oil, mandarin oil, orange oil, oregano oil, lavender oil, Lindera oil, pine needle oil, pepper oil, rose oil, iris oil, sweet orange oil, tangerine oil, tea tree oil, tea seed oil, thyme oil, thymol oil, garlic oil, peppermint oil, onion oil, linaloe oil, Japanese mint oil, spearmint oil and others as disclosed herein throughout.

5. Formulation according to claim 4, further comprising a fragrance.

6. Method of inhibiting malodor in fabrics, textiles or clothing, comprising applying the formulation of any of claims 3-5 directly to a fabric, textile or clothing prior to use.
